(19) United States
(12) Patent Application Publication
(10) Pub. No.: US 2005/0121348 A1
(43) Pub. Date: Jun. 9, 2005

(54) Package insert and stackable package for articles

(76) Inventors: Timothy P. Clare, Appleton, WI (US); David E. Johnson, Neenah, WI (US)

Correspondence Address: KIMBERLY-CLARK WORLDWIDE, INC., 401 NORTH LAKE STREET, NEENAH, WI, 54956, US

(21) Appl. No.: 10/732,758

(22) Filed: Dec. 9, 2003

(51) Int. Cl. A61L 15/00

(52) U.S. Cl. 206/440; 206/449

(57) ABSTRACT

A package for articles includes a structural insert for supporting a stack of articles, and a flexible outer covering containing the insert and the articles. The insert has a base and two opposed side walls extending from the base to define a generally U-shaped channel for receiving and supporting the stack of articles. Each side wall of the insert includes at least one cut-out for reducing the mass and bulk of the insert while retaining sufficient strength for stacking the package above and below other such packages.

### FIELD OF THE INVENTION

The present invention relates to the packaging of flexible articles such as disposable absorbent products. More specifically, the invention relates to a stackable package for shipping, storing and distributing disposable absorbent articles.

### BACKGROUND OF THE INVENTION

Flexible packages for absorbent articles such as infant diapers, training pants, feminine care products, adult incontinence garments, and the like are known. These packages generally include a flexible outer covering in the form of a packaging bag or a flexible film for containing the absorbent articles. For example, absorbent articles may be packaged in packaging bags formed from a polymeric material resulting in a package having a carton-like appearance. In other known packages, a flexible film of thermoplastic material is used to cover a stack of flexible articles which are held in a compressed condition by a paper wrapping. Generally, these types of flexible packages are relatively compact, lightweight, and cost effective. However, these packages usually do not offer rigid protection for the packaged articles and tend to deform when subjected to loads during shipping, storage and distribution. These packages also tend to lack sufficient rigidity to allow for effective stacking during shipping or storage, and compression of lower packages in a stacked pile of such packages can cause the pile to lean.

As a result, many solutions to these problems have been attempted, including forming packages with a rigid packaging support and a flexible film covering. One known package of this type includes a rectangular sleeve of corrugated fiberboard enclosing a plurality of cartons, and a band of heat shrinkable, transparent film disposed around at least a portion of the fiberboard sleeve. In another known package, a five sided rectangular insert is used to support smaller containers, and the containers and insert are enclosed in a heat shrinkable packaging film. It is also known to fasten together pre-cut corrugated cardboard strips to form a rectangular frame, load the frame, and enclose the loaded frame in a flexible plastic film wrap. Other known packages are formed by wrapping a corrugated packaging material including fluting attached to one or more sheets of heat-shrinkable, polymeric film around a series of containers, and heat shrinking the final assembly. Packages with these kinds of rigid supports usually are stackable, and also provide rigid protection for the packaged articles. Nonetheless, these solutions have not always been completely satisfactory. For example, the rigid packaging support tends to be a relatively large component of the package, and consequently contributes to the overall mass, bulk and cost of the packaging.

Accordingly, there remains a need in the art for a package for flexible articles such as disposable absorbent products which is relatively compact, lightweight, and cost effective, and which has sufficient rigidity for stacking.

There is also a need for a compact and lightweight package for disposable absorbent products which includes a protective insert for protecting the articles from damage during shipping, storing and distribution.

### SUMMARY OF THE INVENTION

In one aspect, the present invention provides a package of flexible articles adapted to be stacked vertically upon and beneath other such packages. The package includes a plurality of flexible articles, and a structural insert containing the flexible articles. The insert includes a base and two opposed side walls extending from the base to define a generally U-shaped channel for receiving the flexible articles, each side wall extending from the base to a free edge spaced from the base, and each side wall including at least one cut-out extending into the wall from the free edge thereof. In addition, the package includes a flexible outer covering containing the insert and the flexible articles.

Desirably, each side wall of the package insert includes multiple cut-outs which form a plurality of evenly spaced-apart support ribs in an operatively upper portion of the side wall. Typically, the support ribs have the same shape as adjacent cut-outs, and may, for example, have the shape of a rectangle, a square, a triangle, or a truncated triangle. Alternatively, the support ribs may be curvilinear.

The area defined by the cut-outs may be greater than 10%, desirably is greater than 20%, and most desirably is about 30%, of the total area of the package insert. The total area of the package insert is defined by the area of the base and the two opposed side walls, including the area of the cut-outs.

The structural insert may be formed from corrugated board, such as corrugated cardboard or fiberboard, in which case the side walls may be joined to the base along fold lines.

The absorbent articles may be disposable absorbent articles such as training pants, infant diapers, feminine care products, incontinence products, disposable apparel, or the like.

Typically, the flexible film is a plastic covering in the form of a polymer film. The polymer film may include any suitable polymeric material, and desirably includes a thermoplastic film such as, for example, a polyethylene film or film laminate. The flexible film may also be a shrinkable packaging film such as a polymeric heat shrinkable film.

In one arrangement, the plurality of flexible articles is packaged within at least one flexible covering such as, for example, packaged diapers or training pants. In this arrangement, the packaged articles may be packed within at least one packaging bag or flexible film.

In another aspect of the invention, there is provided a package insert for supporting a plurality of flexible articles within a flexible outer covering wrapped around the insert and the flexible articles. The package insert includes a base and two opposed side walls extending from the base to define a generally U-shaped channel for receiving the flexible articles. Each side wall extends from the base to a free edge spaced from the base, and each side wall includes at least one cut-out extending into the wall from the free edge thereof.

The invention further provides a blank for forming an insert of the type described above. The blank has a base panel including a first side edge and a second side edge. A first side wall panel is joined to the first side edge of the base panel, and a second side wall panel is joined to the second side edge of the base panel. The first side wall panel includes a free edge spaced from the first side edge of the base panel, and at least one cut-out extending into the first side wall panel from the free edge thereof. The second side wall panel includes a free edge spaced from the second side edge of the base panel, and at least one cut-out extending into the second side wall panel from the free edge thereof.

The invention extends to a method for forming a plurality of inserts of the type described above. The method includes forming a first pair of spaced-apart fold lines on sheet material to define side edges of bases for a first set of package inserts, and forming a second pair of spaced-apart fold lines on the sheet material to define side edges of bases for a second set of package inserts. The second pair of fold lines is spaced laterally from the first set of fold lines, and the method includes the steps of cutting the sheet material along undulating lines adjacent the pairs of fold lines to form side walls for the package inserts between the fold lines and a plurality of cut edges. At least one of the cut edges defines support ribs on side walls of one set of package inserts which alternate with nested support ribs on side walls of a laterally adjacent set of package inserts.

In another aspect, the present invention provides a package of flexible articles adapted to be stacked vertically upon and beneath other such packages. The package includes a plurality of flexible articles, and a structural insert containing the flexible articles. The insert includes a base and two opposed side walls extending from the base to define a generally U-shaped channel for receiving the flexible articles, each side wall extending from the base to a free edge spaced from the base, and each free edge defining an undulating pattern. In addition, the package includes a flexible outer covering containing the insert and the flexible articles.

The broad scope of the applicability of the present invention will become apparent to those of skill in the art from the details given below.

The detailed description of the preferred embodiments of the invention is given by way of example only, and various modifications within the scope of the invention will be apparent to those of skill in the art.

### BRIEF DESCRIPTION OF THE DRAWINGS

FIG. 1 is a perspective view of a stack of absorbent articles.

FIG. 2 is a perspective view of a package insert according to a first aspect of the present invention.

FIG. 3 is a perspective view of a package of absorbent articles according to the present invention.

FIG. 4 is a top view of a blank for forming the package insert of FIG. 2.

FIG. 5 is a top view of a portion of sheet material for forming the blank of FIG. 4.

FIG. 6 is a perspective view of a package insert according to a second aspect of the invention.

FIG. 7 is a perspective view of a package insert according to a third aspect of the invention.

FIG. 8 is a perspective view of a package of absorbent articles according to another aspect of the invention.

FIG. 9 is a perspective view of a package of absorbent articles according to yet another aspect of the invention.

### DEFINITIONS

As used herein, the term “disposable” when used to describe an article or a product, means garments or articles which are designed to be discarded after a limited use rather than being laundered or otherwise restored for use.

As used herein, the term “flexible” is intended to mean compliant in the sense of yielding under a load.

As used herein, the term “rigid” is intended to mean stiff in the sense of having the capacity to receive a load.

As used herein, the terms “comprises” and “comprising” have the same meaning as the terms “includes” and “including” and are intended to be inclusive or open-ended so as not to exclude additional elements or method steps. For example, the term “comprising” specifies the presence of the stated features, elements, integers, steps or components, but does not preclude the presence or addition of one or more other features, elements, integers, steps, components or groups thereof.

### DETAILED DESCRIPTION

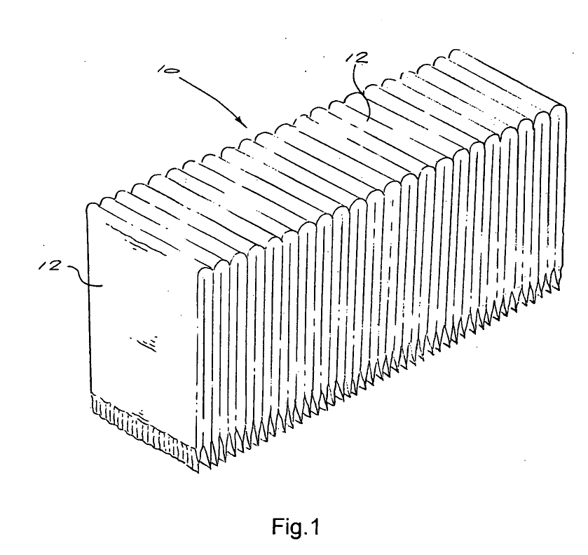

FIG. 1 of the drawings illustrates a stack 10 of folded absorbent articles 12 for a bundled package according to the present invention. The absorbent articles 12 may be disposable training pants such as the training pants described in U.S. Pat. No. 4,940,464 to Van Gompel et al and U.S. Pat. No. 6,645,190 to Olson et al, the contents of which are incorporated herein by reference to the extent that they are consistent with the current disclosure. Other flexible articles which are suitable for bundled packages and which could be bundled into a package according to the present invention include, but are not limited to, non-disposable apparel, tissue products, infant diapers, feminine care products, incontinence products, disposable apparel, and the like.

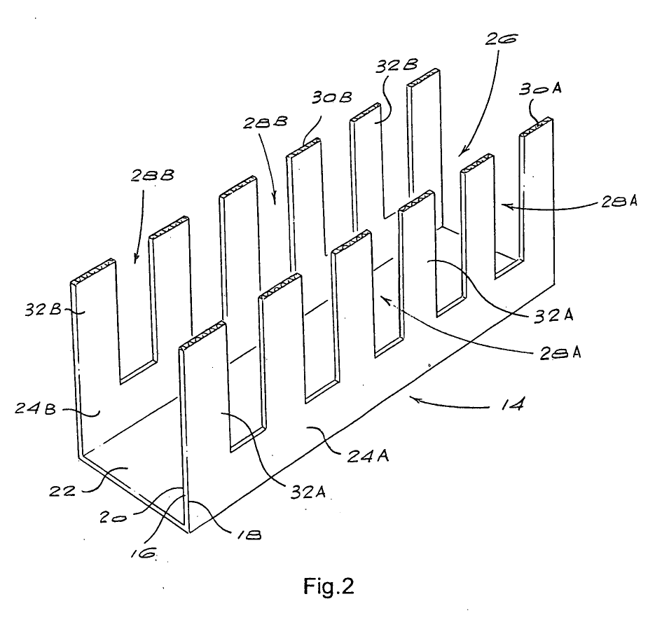

FIG. 2 illustrates an insert 14 for supporting the absorbent articles 12 in a manner which is described in more detail below. The insert 14 may be formed from various materials having suitable support characteristics. For example, as representatively illustrated in FIG. 2, the insert 14 may be formed from corrugated cardboard. Alternatively, the insert 14 may be formed from various other materials, including non-corrugated cardboard, fiberboard, paper and generally any board-like material made from paper or its derivatives, rigid materials made from rubber bases, plastic, or the like, or combinations thereof. In a particular aspect, the insert 14 may include a corrugated or fluted sheet 16 bonded between a pair of spaced walls 18 and 20, and typically is formed so that the fluting is vertically oriented, in use.

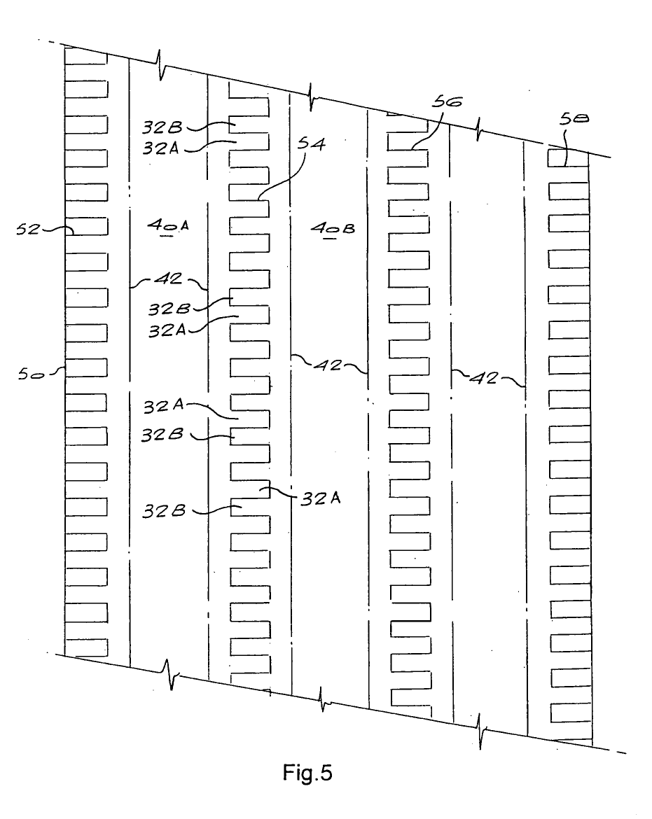

As can be seen, the insert 14 may include a base 22 and a pair of opposed side walls 24A and 24B which define a generally U-shaped channel 26 for receiving and supporting the stack of absorbent articles 12 illustrated in FIG. 1. The side walls 24A and 24B may include a plurality of evenly spaced-apart cut-outs 28A and 28B which extend into the side walls from operatively upper free edges 30A and 30B, as shown. As such, the upper free edges 30A and 30B define an undulating pattern. In the illustrated aspect, each cut-out 28A is spaced from an adjacent cut-out by a support rib 32A formed from an upper portion of the side wall 24A, and each cut-out 28B is spaced from an adjacent cut-out by a support rib 32B formed from an upper portion of the side wall 24B. In the aspects of the invention illustrated in FIGS. 2 and 5, the cut-outs 28A are evenly spaced, rectangular in shape and form mirror images of adjacent support ribs 32A, and the cut-outs 28B are rectangular in shape and form mirror images of adjacent support ribs 32B. Thus, the undulating pattern of the free edges 30A and 30B may include the spaced support ribs 32A. In other aspects of the invention, the cut-outs may be staggered or randomly located in the side walls. Typically, the area displaced by the cut-outs is greater than about 10% of the total area of the insert 14. In a particular aspect of the invention, the area displaced by the cut-outs is greater than about 20%, and desirably is equal to about 30%, of the total area of the insert 14. The total area of the insert is calculated by adding the area of the base 22 to the area of the two opposed side walls 24A and 24B, including the area of the cut-outs 28A and 28B.

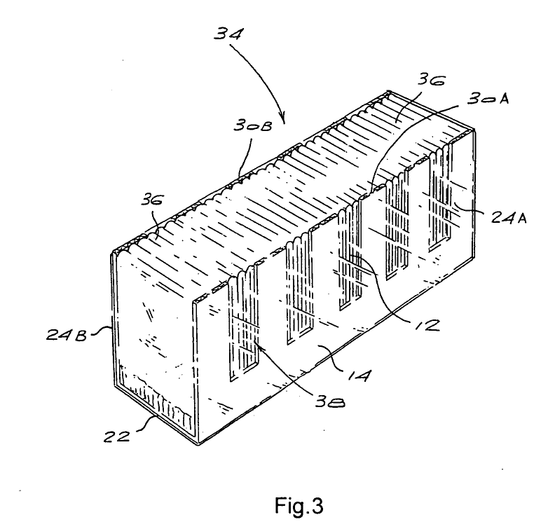

Turning now to FIG. 3, there is illustrated a bundled package according to one aspect of the present invention. The package is designated generally with the reference numeral 34 and includes the stack of absorbent articles of FIG. 1 supported within the generally U-shaped channel of the package insert of FIG. 2. As representatively illustrated, each absorbent article 12 may extend from the base 22 of the insert 14 to the upper edges 30A and 30B of the side walls 24A and 24B so that the folded edges 36 of the absorbent articles 12 are substantially flush with the upper edges 30A and 30B of the insert. In another arrangement, the absorbent articles 12 may extend beyond the upper edges 30A and 30B of the side walls 24A and 24B, or alternatively, the upper edges 30A and 30B of the side walls 24A and 24B may extend beyond the absorbent articles 12. A flexible film 38 contains the insert 14 and the articles 12. For example the film 38 may be wrapped around the insert 14 and the absorbent articles 12 to form an outer covering for the package 34. The film may be transparent, as illustrated, or it may be translucent, opaque, coloured, or combinations thereof.

In one aspect of the invention, the flexible film 38 may be a polymer film having sufficient flexibility to assume a desired, generally hexahedral shape when wrapped around the insert 14 and the absorbent articles 12, and having sufficient strength to hold and contain the absorbent articles 12 within the insert 14 without breaking and without excessive bulging or stretching. The polymer film 38 may include any suitable polymeric material. For example, the film may be a thermoplastic film such as a polyethylene film or film laminate having a thickness of about 2.5 mils (about 0.0635 millimeters). The polyethylene film or film laminate may be a low density polyethylene (LDPE) film, a linear low density polyethylene (LDPE/LLDPE) film laminate, a medium density polyethylene (LDPE/MDPE) film laminate, a high density polyethylene (LDPE/HDPE) film laminate, or the like. The film may be applied using compression wrapping such as STARWRAP wrapping available from Gevas Ltd having offices in Boynton Beach, Fla., United States of America. Other materials that could be used for the film or film laminate include polyester, polypropylene or combinations or laminates of these. The flexible film may also be a heat shrinkable packaging film formed from, for example, cross-linked and oriented polyethylene and polypropylene, polyisopropylethylene or polyisobutylethylene. The use of polymeric heat shrinkable films for the packaging of materials is well known in the art, and is described, for example, in U.S. Pat. No. 3,050,402 to Dreyfus et al, U.S. Pat. No. 3,198,327 to Boehling et al, and U.S. Pat. No. 3,447,675 to Kirby.

It will be understood that the insert 14 provides rigidity for the package 34 so that the package can be effectively stacked upon and below other such packages during shipping, storage and distribution. In this regard, when the package 34 is stacked, the side walls 24A and 24B receive loads from packages above the insert 14 and transfer these loads to packages below the insert, thereby resisting distortion or deformation of the package and consequential leaning of a pile of stacked packages. Also, the base 22 of the insert 14 provides a rigid support for the bottom of the package 34 which protects the bottom of the package from damage on a conveyor. The insert 14 also provides sufficient rigidity for use of compression wrapping, such as STARWRAP wrapping, without adverse deformation of the absorbent articles. It will also be appreciated that the cut-outs 28A and 28B in the side walls 24A and 24B, and the absence of end walls on the insert 14, allow the package 34 to be relatively lightweight, compact and cost-effective. Furthermore, the cut-outs 28A and 28B in the side walls 24A and 24B, and the lack of end walls on the insert 14, allow the articles 12 to be viewed through the flexible film 38. Accordingly, graphics or the like on the absorbent articles 12 could be displayed, if desired.

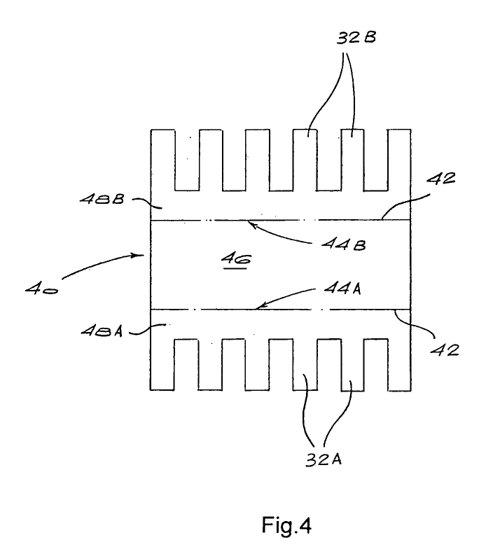

FIG. 4 illustrates a blank 40 for forming the package insert 14. The blank 40 may be formed from corrugated cardboard and is seen to include a pair of spaced-apart score lines 42 defining side edges 44A and 44B of a base panel 46. Side wall panels 48A and 48B extend laterally from the side edges 44A and 44B so as to be foldable about the score lines 42 to form the side walls of the insert 14. As can be seen, the side wall panels 48A and 48B may be cut along outer edges thereof to define the support ribs 32A and 32B on the insert 14.

With reference now to FIG. 5 of the drawings, a plurality of blanks for the insert 14 may be formed by cutting a sheet of insert material 50 such as corrugated cardboard. In this aspect of the invention, the method of forming the blanks includes creating a plurality of the score lines 42, as shown, and cutting the insert material 50 adjacent each pair of score lines 42 along undulating lines 52, 54, 56 and 58. Each cut along the lines 52 to 58 defines the support ribs on one set of blanks which alternate with nested support ribs on a laterally adjacent set of blanks. For example, the cut along the line 54 defines support ribs 32A on one set of blanks 40A which alternate with nested support ribs 32B on a laterally adjacent set of blanks 40B. In this way, the blanks are formed economically with relatively little waste. The insert material 50 is also cut transversely to the lines 52 to 58 to form suitable lengths of blanks for the inserts 14.

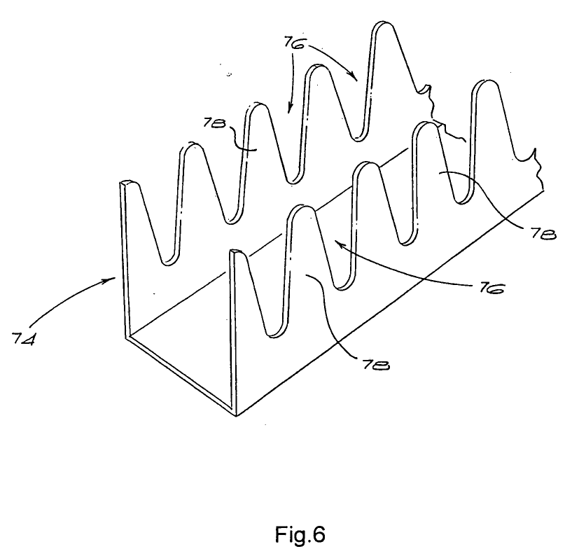

FIG. 6 illustrates a package insert 68 according to a second aspect of the invention. The insert 68 may be formed from corrugated cardboard or other suitable materials as described above, and is similar to the insert 14 in all respects except for the shapes of the cut-outs and the support ribs. In this aspect of the invention, each cut-out 70 and each support rib 72 has the shape of a truncated triangle instead of a rectangle. It will be appreciated that, since the shape of the support ribs 72 in FIG. 6 is identical, but for being inverted, to the shape of the cut-outs 70, a blank for forming the insert 68 could be formed in a similar way to that described immediately above with reference to the insert 14.

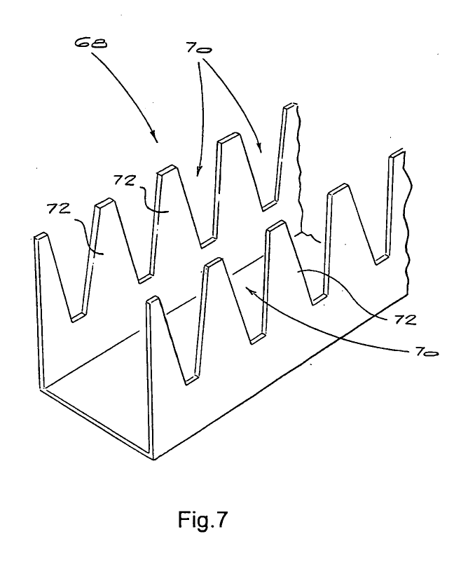

FIG. 7 illustrates a packaging insert 74 according to a third embodiment of the invention in which the cut-outs 76 and the support ribs 78 are formed from curved, undulating lines. Apart from the shapes of the cut-outs and the support ribs, the insert 74 is similar in all respects to the insert 14 of the first embodiment of the invention.

It will be appreciated that the invention is not limited to the specific cut-outs described above with reference to FIGS. 2, 4, 6 and 7 of the drawings, and that various differently shaped cut-outs could be used to provide similar advantages. For example, the cut-outs could be square, triangular or arcuate in shape, or combinations thereof. Also, although the invention has been described above with reference to a stack of absorbent articles which are located within a packaging insert prior to wrapping, it will be appreciated that the stack of absorbent articles could be compressed prior to being inserted into the packaging insert.

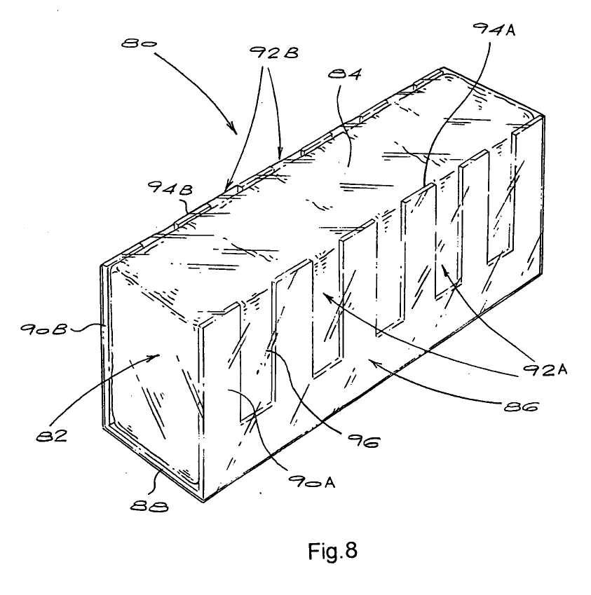

FIG. 8 illustrates a bundled package 80 according to another aspect of the invention. In this aspect, the package 80 includes articles packaged in at least one flexible covering, such as, for example, packaged training pants. The packaged articles may be pre-packages and are designated generally with the reference numeral 82 and may include a stack of absorbent articles (not visible) contained within at least one flexible outer covering in the form of a packaging bag 84. Conventional packaging bags of this type often are used to supply disposable absorbent articles such as training pants and the like. Examples of such packaging bags include those described in U.S. Pat. No. 5,967,665 to MacDonald et al and U.S. Pat. No. 6,318,555 to Kuske et al, the contents of which are incorporated herein by reference to the extent that they are consistent with the current disclosure. Apart from the packaged articles 82, the package 80 is similar in all respects to the package 34 described above. Accordingly, the package 80 includes a rigid support insert 86 similar in all respects to the insert 14 described above. As can be seen, the insert 86 includes a base 88 and a pair of opposed side walls 90A and 90B which define a generally U-shaped channel for receiving and supporting the pre-packaged articles 82. The side walls 90A and 90B include a plurality of evenly spaced-apart cut-outs 92A and 92B which extend into the side walls from upper free edges 94A and 94B thereof, as shown. A flexible film 96, similar to the flexible film 38 described above, is wrapped around the pre-packaged articles 82 and the insert 86 to form an outer covering for the package 80.

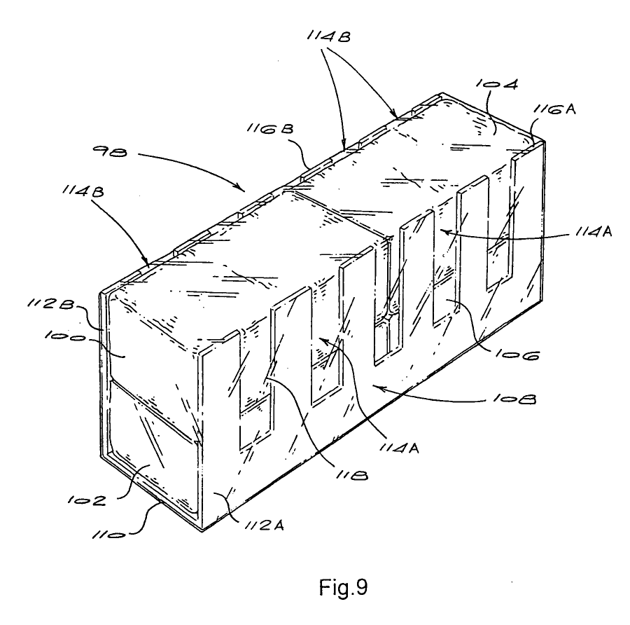

As may be readily appreciated by one of skill in the art, a plurality of packages of pre-packaged articles may be bundled within a rigid support insert of the present invention. For example, as representatively illustrated in FIG. 9 the package 98 includes four packaging bags 100, 102, 104 and 106 containing pre-packaged articles such as, for example, pre-packaged training pants or the like. Alternatively, fewer than four packaging bags or greater than four packaging bags may be included within the package 98.

The packaging bags 100, 102, 104 and 106 are all similar to the packaging bag 84 described above with reference to FIG. 8 of the drawings, and apart from the number of packaging bags, the package 98 is similar in all respects to the package 80. Thus, the package 98 includes a rigid support insert 108 which includes a base 110 and a pair of opposed side walls 112A and 112B defining a generally U-shaped channel for receiving and supporting the packaging bags 100, 102, 104 and 106. When multiple packaging bags are located within a package 98, the packaging bags 100, 102, 104, and 106 may be arranged in numerous configurations as are known in the art. For example, the packaging bags 100, 102, 104 and 106 may be oriented as shown in FIG. 9, or alternatively may all be located in the same plane, without any stacking within the support insert 108.

The side walls 112A and 112B include a plurality of evenly spaced-apart cut-outs 114A and 114B which extend into the side walls from upper free edges 116A and 116B thereof. A flexible film 118, similar to the flexible film 38 described above, is wrapped around the packaging bags 100, 102, 104 and 106 and around the insert 108 to form an outer covering for the package 98.

It will be appreciated that the pre-packaged articles referred to in FIGS. 8 and 9 need not be training pants, and could be various other types of flexible articles such as, for example, infant diapers, feminine care products, incontinence products, disposable apparel, and the like. It will also be appreciated that the pre-packaged articles could be contained within a flexible outer covering other than a packaging bag, for example a flexible film. Furthermore, it will be understood that the packages 80 and 98 could include more than one insert 86 and 108.

Having thus described the invention in sufficient detail, it should be apparent to those skilled in the art that various modifications can be made without departing from the scope of the invention as defined in the following claims.

1. A package of flexible articles adapted to be stacked vertically upon and beneath other such packages, the package comprising:
a plurality of flexible articles;
a structural insert partially surrounding the flexible articles, the insert comprising a base and two opposed side walls extending from the base to define a generally U-shaped channel for receiving the flexible articles, each side wall extending from the base to a free edge spaced from the base, and each side wall including at least one cut-out extending into the wall from the free edge thereof; and
a flexible outer covering containing the insert and the flexible articles.

2. The package of claim 1, wherein each side wall of the insert includes a plurality of cut-outs extending into the wall from the free edge thereof to form a plurality of evenly spaced-apart support ribs.

3. The package of claim 2, wherein each support rib has the same shape as an adjacent cut-out.

4. The package of claim 3, wherein each support rib has the shape of a rectangle.

5. The package of claim 3, wherein each support rib has the shape of a triangle.

6. The package of claim 3, wherein each support rib has the shape of a truncated triangle.

7. The package of claim 1, wherein each side wall of the insert includes a plurality of cut-outs extending into the wall from the free edge thereof to form a plurality of irregularly spaced-apart support ribs.

8. The package of claim 1, wherein the area defined by the at least one cut-out is greater than 10% of the total area of the insert.

9. The package of claim 8, wherein the area defined by the at least one cut-out is greater than 20% of the total area of the insert.

10. The package of claim 8, wherein the area defined by the at least one cut-out is about 30% of the total area of the insert.

11. The package of claim 1, wherein the insert is formed from a corrugated board.

12. The package of claim 1, wherein the flexible articles are contained within at least one package of articles.

13. The package of claim 1, wherein the flexible articles are absorbent articles.

14. The package of claim 13, wherein the absorbent articles are disposable.

15. The package of claim 14, wherein the absorbent articles are training pants.

16. The package of claim 14, wherein the absorbent articles are infant diapers.

17. The package of claim 14, wherein the absorbent articles are feminine care products.

18. The package of claim 1, wherein the flexible outer cover is a polymer film which is compression wrapped about the flexible articles and the insert.

19. A package insert for supporting a plurality of flexible articles within a flexible outer covering wrapped around the insert and the flexible articles, the package insert comprising;
a base; and
two opposed side walls extending from the base to define a generally U-shaped channel for receiving the flexible articles, each side wall extending from the base to a free edge spaced from the base, and each side wall including at least one cut-out extending into the wall from the free edge thereof.

20. The package insert of claim 23, wherein each side wall of the insert includes a plurality of cut-outs extending into the wall from the free edge thereof to form a plurality of evenly spaced-apart support ribs.

21. The package insert of claim 23, wherein each side wall of the insert includes a plurality of cut-outs extending into the wall from the free edge thereof to form a plurality of irregularly spaced-apart support ribs.

22. The package insert of claim 24, wherein each support rib has the same shape as an adjacent cut-out.

23. The package insert of claim 26, wherein each support rib has the shape of a rectangle.

24. The package insert of claim 26, wherein each support rib has the shape of a triangle.

25. The package insert of claim 26, wherein each support rib has the shape of a truncated triangle.

26. The package insert of claim 23, wherein the area defined by the at least one cut-out is greater than 10% of the total area of the insert.

27. The package insert of claim 30, wherein the area defined by the at least one cut-out is greater than 20% of the total area of the insert.

28. The package insert of claim 30, wherein the area defined by the at least one cut-out is about 30% of the total area of the insert.

29. The package insert of claim 23, formed from a corrugated board.

30. A blank for forming a package insert comprising:
a base panel including a first side edge and a second side edge;
a first side wall panel joined to the first side edge, the first side wall panel comprising a free edge spaced from the first side edge, and at least one cut-out extending into the first side wall panel from the free edge thereof; and
a second side wall panel joined to the second side edge, the second side wall panel comprising a free edge spaced from the second side edge of the base panel, and at least one cut-out extending into the second side wall panel from the free edge thereof.

31. The blank of claim 30, wherein each side wall panel includes a plurality of cut-outs extending into the wall from the free edge thereof to form a plurality of evenly spaced-apart support ribs.

32. The blank of claim 31, wherein each support rib has the same shape as an adjacent cut-out.

33. The blank of claim 30, wherein the area defined by the cut-outs is about 30% of the total area of the blank.

34. A method for forming a plurality of package inserts comprising:
forming a first pair of spaced-apart fold lines on a sheet material to define side edges of bases for a first set of package inserts;
forming a second pair of spaced-apart fold lines on the sheet material to define side edges of bases for a second set of package inserts, the second pair of fold lines being spaced laterally from the first set of fold lines;
cutting the sheet material along undulating lines adjacent the pairs of fold lines to form side walls for the package inserts between the fold lines and a plurality of cut edges, at least one of the cut edges defining support ribs on side walls of one set of package inserts that alternate with nested support ribs on side walls of a laterally adjacent set of package inserts.

35. The method of claim 34, wherein the at least one cut along the undulating lines forms support ribs in the side walls of one set of package inserts which have the same shape as alternating, nested support ribs in the side walls of a laterally adjacent set of package inserts.

36. The method of claim 34, further comprising the step of cutting the sheet material transversely with respect to the fold lines so as to separate each package insert in a set of inserts from adjacent package inserts in the set.

37. The method of claim 36, further comprising the step of folding the cut side walls of each package insert relative to the base thereof, along the fold lines, to define a generally U-shaped channel.

38. A package of flexible articles adapted to be stacked vertically upon and beneath other such packages, the package comprising:
a plurality of flexible articles;
a structural insert partially surrounding the flexible articles, the insert comprising a base and two opposed side walls extending from the base to define a generally U-shaped channel for receiving the flexible articles, each side wall extending from the base to a free edge spaced from the base, and each free edge defining an undulating pattern; and
a flexible outer covering containing the insert and the flexible articles.

39. The package of claim 38, wherein the undulating pattern comprises a plurality of evenly spaced-apart support ribs.

40. The package of claim 39, wherein each support rib has the same shape as an adjacent support rib.

41. The package of claim 40, wherein each support rib has the shape of a rectangle.

42. The package of claim 40, wherein each support rib has the shape of a triangle.

43. The package of claim 40, wherein each support rib has the shape of a truncated triangle.
